(19) United States
(12) Patent Application Publication
(10) Pub. No.: US 2017/0035960 A1
(43) Pub. Date: Feb. 9, 2017

(54) MECHANICALLY ACTUATED INFUSION DEVICE HAVING DOSE COUNTER

(71) Applicant: LifeScan Scotland Limited, Inverness (GB)

(72) Inventor: David ELDER, Inverness (GB)

(21) Appl. No.: 14/818,378

(22) Filed: Aug. 5, 2015

(51) Int. Cl. A61M 5/142; A61M 5/172; A61M 5/168

(52) U.S. Cl. CPC A61M 5/14244; A61M 5/168; A61M 5/172; A61M 2005/14208; A61M 2205/52

(57) ABSTRACT

A mechanically operated medical infusion device with a dose counter is disclosed herein. The infusion device includes a pump and at least one mechanical activation mechanism for mechanically engaging the pump to deliver a dose of medicament to cause a dose event. The dose counter includes a sensor for detecting a vibration signature indicative of the dose event and a micro-controller for recording the dose event.

### TECHNICAL FIELD

This application generally relates to the field of medicament delivery systems and more specifically to a mechanically operated medical infusion device that includes a dose counter.

### BACKGROUND

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses (boluses) for meals and snacks. These infusion systems have shown to improve control of blood glucose levels. However, they suffer the drawbacks of size, cost, and complexity. For example, these pumps are electronically controlled and must be programmed to supply the desired amounts of basal and bolus insulin. This prevents many patients from accepting this technology over the standard subcutaneous injections.

Thus, a number of highly compact mechanical solutions, such as the Calibra Finesse© insulin patch pump, have been create to provide a convenient form of insulin treatment which does not require significant programming or technical skills to implement to service both basal and bolus needs has been developed. Such an infusion device is simple to use and mechanically driven, negating the need for batteries and the like and enabling a very compact design. The infusion device can be directly attached to the body and does not require any electronics in order to program the delivery rates. The insulin is preferably delivered through a small, thin-walled tubing (cannula) through the skin into the subcutaneous tissue similar to technologies in the prior art.

Historical information indicating when a patient received a dose is important in managing chronic conditions and diseases, such as diabetes. Insulin-dependent diabetics, for example, need to know how much insulin they have injected into their body and when, so that they can determine how much insulin they should receive to compensate for meals, etc. However, because these compact insulin delivery devices are purely mechanical, there is no way of storing dosing information. Dose counting devices that provide means for tracking the number of doses of medication delivered to a patient have been proposed. However, these dose counting devices do not includes means for including a timestamp in the dosing information. In addition, the proposed dose counting devices are typically integrated with the infusion devices, which are typically disposable, and are thus not reusable.

### BRIEF DESCRIPTION OF THE INVENTION

Various embodiments of a dose counter for a mechanically operated medical infusion device are described herein. Advantageously, the dose counter includes a method for adding a timestamp to dosing information. In addition and according to at least one version, the dose counter can be removably coupled to the infusion device, allowing the dose counter to be reusable.

In a first aspect, an infusion device is described. The infusion device includes a reservoir that holds a liquid medicament and a pump that displaces a volume of the liquid medicament when mechanically activated by an activation mechanism, such as, for example, by the muscles of a user, in order to produce a dose event. The infusion device also includes a dose counter. The dose counter includes a sensor configured to detect vibrations indicative of operation of the activation mechanism and a micro-controller coupled to the sensor.

The dose counter can further include a micro-controller coupled to the sensor to record the dose event. The activation mechanism can be at least one depressible button. The at least one depressible button generates a vibration signature indicative of a dose event. The micro-controller is configured to distinguish the vibration signature from an incidental vibration. The micro-controller can further include a real-time clock and a memory to store a record and a timestamp of each dose event. The micro-controller can be configured to prevent counting a dose event for a predetermined period of time after determination of the dose event in order to prevent counting the dose event more than once. The infusion device can further include a portable power device. The dose counter can further include a communication interface configured to transmit the time of each activation of the pump. The dose counter can be removably coupled to the infusion device.

According to another aspect, a dose counter for a mechanically operable infusion device is described. The infusion device includes a pump and at least one mechanical activation mechanism for engaging the pump to produce a dose event. The dose counter includes at least one sensor that is configured to detect vibrations that are indicative of the dose event and a micro-controller. The micro-controller can include a clock and a memory for storing the data obtained by the at least one sensor. In at least one embodiment, the micro-controller is configured to record occurrence of each dose event as well as a timestamp indicative of each dose event for storage in which the indication of the dose event can be transmitted.

The dose counter can further include a portable power source configured to power the dose counter. According to one version, the at least one sensor can be an electret microphone. The dose event generates a signature vibration detected by the at least one sensor. The dose counter can further include a communication interface and the micro-controller is configured to transmit a record of each dose event using the interface. In at least one embodiment, the communication interface includes a near field communication (NFC) interface. The micro-controller can further include a counter configured to record occurrence of each dose event.

According to yet another aspect, a method for determining a dose event of an infusion device is described. The infusion device includes a pump and at least one mechanically actuated mechanism for engaging the pump to cause a dose event. In addition, the infusion device includes a sensor. The method includes detecting vibrations using the sensor generated by engaging the at least one activation mechanism to cause the dose event. The method additionally includes determining whether the vibrations constitute an actual dose event.

The method can further include advancing a counter to record occurrence of the dose event. Additionally, the method can include recording a timestamp of the dose event. The sensor can include at least one determining sensor capable of detecting the vibration coupled to a micro-controller that is configured to make a determination based on sensor input. In at least one embodiment, the at least one sensor is an electret microphone. The method can further include transmitting the recorded time of the dose event. In another embodiment, the method further includes activating a temporary counting lock-out system configured to prevent counting a dose event more than once. In at least one embodiment, the activation mechanism is at least one depressible button. The at least one depressible button generates a vibration signature indicative of a dose event. The micro-controller is configured to distinguish a vibration signature indicative of a dose event from an incidental vibration. In at least one embodiment, the sensor is a sensor module removably coupled to the infusion device. In another embodiment, the sensor is integrally provided.

One advantage realized is that a compact and mechanically operated infusion device can be configured to perform dose counting, recording the dose counting, including a timestamp, and transmission of the recorded dose counting.

Another advantage is that the dose counter can be removably coupled to a infusion device, resulting in a dose counter that can be removed from the infusion device and reused.

These and other features and advantages will become apparent to those skilled in the art when taken with reference to the following more detailed description of the exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 is a perspective view of a mechanically operated infusion device including a dose counter in accordance with an exemplary embodiment;

FIG. 2 is a schematic representation of the valves and pump of the infusion device of FIG. 1;

FIG. 3 is an exploded assembly view of the infusion device of FIG. 1;

FIG. 4 is a functional block diagram of a dose counter in accordance with an exemplary embodiment;

FIG. 5 illustrates a waveform of a recognizable vibration signature representative of a dose event;

FIG. 6 illustrates a waveform of an incidental vibration;

FIG. 7 illustrates waveforms of exemplary vibration signatures;

FIG. 8 is a schematic diagram of a sensor used for detecting dose events and in accordance with an exemplary embodiment; and

FIG. 9 is a flowchart depicting an exemplary method of recording a dose event.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the intended scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

The term “medicament” means a volume of a liquid, solution or suspension, intended to be administered to a patient. As used herein, the terms “comprising”, “comprise” and “comprises” are open-ended terms intended not to be fully inclusive and in which the terms “include”, “including” and “includes” are intended to have the same intent. While the device(s) are herein described as having “one” part or component, it is to be understood that the term “one” implicitly refers to “at least one”.

The terms “about” and “substantially” are used in connection with a numerical value throughout the description and claims denote an interval of accuracy, familiar and acceptable to a person skilled in the art. The interval governing this term is preferably ±20%. Unless specified, the terms described above are not intended to narrow the scope of the invention as described herein and according to the claims.

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

As will be discussed in more detail below, the disclosed systems and methods relate to a mechanically operated medical infusion device having a pump and at least one activation mechanism, such as a depressible button, for engaging the pump to cause a dose event. A dose counter provided for the device includes a sensor that is configured to detect a vibration signature representative of the dose event.

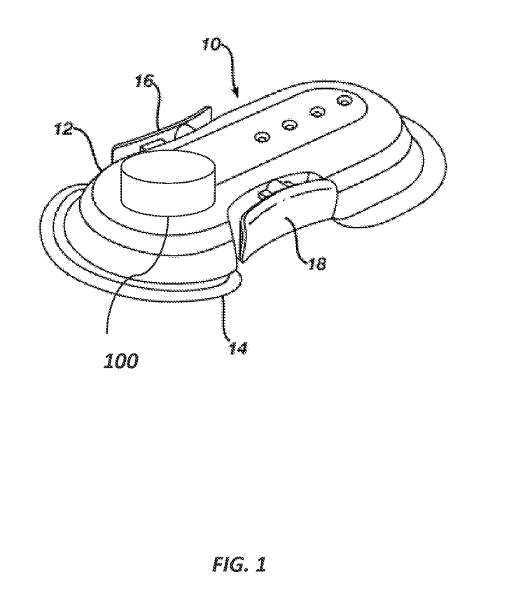

FIG. 1 depicts a perspective view of an infusion device. The infusion device 10 generally includes an enclosure 12, a base 14, a first activation mechanism 16, and a second activation mechanism 18. In the depicted example, the first activation mechanism 16 and the second activation mechanism 18 are depressible buttons disposed on opposing sides of the enclosure 12. In use, the activation mechanisms 16, 18 are each configured to toggle between a first, non-actuated position and a second, actuated position. It is to be understood that while the infusion device 10 is illustrated herein as including two (2) activation buttons, this parameter can be suitably varied. For example, the infusion device 10 can include at least one activation mechanism.

According to this version, the enclosure 12, as will be seen subsequently, is formed by a series of multiple device layers being brought together. Each device layer defines various components of the device such as, for example, a reservoir, various fluid conduits, pump chambers, and valve chambers. This form of device construction, in accordance with aspects of the present invention, enables manufacturing economy to an extent rendering the device disposable after intended use by a patient.

The base 14 preferably includes an adhesive coating (not shown) to permit the device 10 to be adhered to a patient's skin. The adhesive coating may originally be covered with a releasable cover (not shown) that may be peeled from the base 14 when the patient endeavors to deploy the device 10 and attach the device 10 to the skin of the patient. Such arrangements are well known in the art.

The infusion device 10 may be mated with a previously deployed cannula assembly. However, it is contemplated herein that the various aspects of the present invention may be realized within a device that may be alternatively first adhered to the patient's skin followed by the deployment of a cannula thereafter.

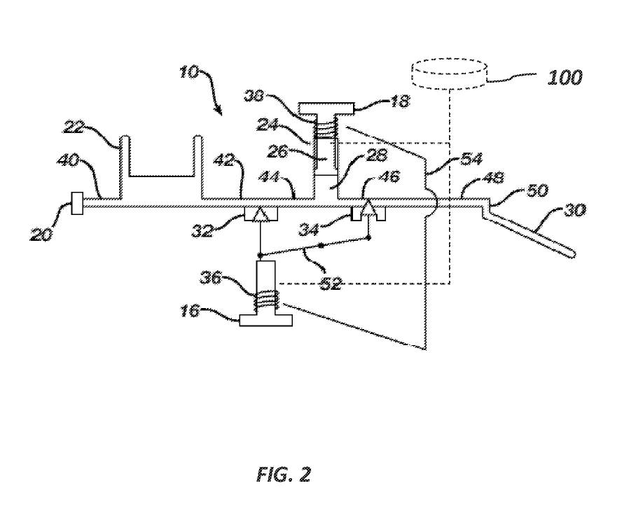

As noted, the activation mechanisms 16 and 18 are placed on opposite sides of the device 10 and directly across from each other. This positioning more readily insures the concurrent depression of the buttons when the patient wishes to receive a dose (bolus) of the liquid medicament contained within the device 10. This arrangement also imposes substantially equal and opposite forces on the device 10 during dosage delivery to prevent the device 10 from being displaced and possibly stripped from the patient. As will be further seen hereinafter, the concurrent depression of the buttons 16, 18 is used to particular advantage. More specifically, the activation mechanism 16 may serve as a valve control which, when in a first position as shown in FIG. 2, establishes a first fluid path between the device reservoir and the device pump to support pump filling, and then, when in a second or depressed position, establishes a second fluid path between the device pump and the device outlet or cannula to permit dosage delivery to the patient. In addition, a linkage between the control activation mechanisms 16 and 18 permits actuation of the device pump with the second activation mechanism 18 only when the second fluid path has been established by the first activation mechanism 16. Hence, the first activation mechanism 16 may be considered a safety control. Additional details regarding the features of the exemplary infusion device can be found in pending U.S. patent application Ser. No. 14/289,930, entitled “Manually Actuated Infusion Device and Dose Counter,” published as U.S. Patent Application Publication No. 2014/0378903A1 and U.S. Pat. No. 7,976,500, entitled “Disposable Infusion Device with Redundant Valved Safety,” the entirety of each document being incorporated herein by reference.

The infusion device 10 further includes a dose counter 100. The dose counter 100 is configured to record dose events and includes a sensor configured to detect the occurrence of dose events. In at least one embodiment, described further below, the sensor detects a vibration signature indicative of the occurrence of the dose event based on engagement of the activation mechanisms by the patient. In an embodiment, the dose counter 100 can be an add-on device or module that is removably coupled to the infusion device 10. Alternatively, the dose counter 100 can be integral with and manufactured as part of the infusion device 10. The method of attachment of the dose counter 100 as a module can include hook and loop fasteners, adhesives, latches, snap-fit, or other suitable means that permits secure but releasable attachment.

FIG. 2 provides a schematic representation of the fluidic system employed by of the infusion device 10 of FIG. 1. More specifically, the infusion device 10 further includes a fill port 20, a reservoir 22, a pump 24, and the cannula 30. The infusion device 10 further includes a first valve 32 and a second valve 34. A plurality of fluid conduits are provided. More specifically and according to this version, a fluid conduit 40 provides a fluidic connection between the fill port 20 and the reservoir 22, fluid conduit 42 provides a fluidic connection between the reservoir 22 and the first valve 32, fluid conduit 44 provides a fluidic connection between the first valve 32 and the pump 24, fluid conduit 46 provides a fluidic connection between the pump 24 and the second valve 34, and fluid conduit 48 provides a fluidic connection between the second valve 34 and the device outlet 50. The outlet 50 is arranged to communicate with the cannula 30.

As shown, the activation mechanisms 16 and 18 of this infusion device 10 are spring-loaded or biased by springs 36 and 38. The springs 36, 38 are provided for returning the activation mechanisms 16, 18 to the first position after a bolus is administered.

The pump 24 of the infusion device 10 comprises a piston pump. The pump 24 includes a pump piston 26 and a pump chamber 28. In accordance with this embodiment, the activation mechanism 18 is directly coupled to and is an extension of the pump piston 26.

With further reference to FIG. 2, the device 10 additionally includes a first linkage 52 and a second linkage 54. The first linkage 52 is a toggle linkage between the first valve 32 and the second valve 34. The first linkage 52 is arranged to assure that the second valve 34 does not open until after the first valve 32 is closed. The second linkage 54 is provided between the first activation mechanism 16 and the second activation mechanism 18. The second linkage 54 is arranged to assure that the pump 24 is not operable until after the first valve is closed and the second valve is opened by the first activation mechanism 16.

Still further, the second valve 34 is a safety valve that closes tighter responsive to increased fluid pressure within the fluid conduit 46. This closure assures that the liquid medicament is not accidentally administered to the patient notwithstanding the inadvertent application of pressure to the reservoir 22, for example. In applications such as this, it is not uncommon for the reservoir 22 to be formed from a flexible material. While this manufacture has its advantages, it does present the risk that the reservoir 22 may be accidentally squeezed as it is worn by the patient. Because the second valve 34 only closes tighter under such conditions, it is assured that increased accidental reservoir pressure will not cause the fluid medicament to flow to the cannula 30.

In operation, the reservoir 22 is first filled through the fill port 20 to a desired level of medicament. In this state, the first and second valves 32 and 34 will be in the positions as shown in which the first valve 32 is open and the second valve 34 is closed. This configuration permits the pump chamber 28 to be filled after the reservoir 22 is filled. The cannula 30 may then be deployed followed by the deployment of the infusion device 10. In this state, the first and second valves 32 and 34 will remain in the depicted configuration with the first valve 32 being open and the second valve 34 closed. This arrangement permits the pump chamber 28 to be filled through a first fluid path, including conduits 42 and 44, as the piston 26 returns to its first position after each applied dose.

When the patient wishes to receive a dose of medicament, the opposing activation mechanisms 16, 18 are simultaneously pressed using mechanical power of the patient's fingers. As used herein, the term “mechanically driven” or “mechanically actuated” indicates that the primary power source is muscle in nature. According to this version of the device 10, the first linkage 52 causes the first valve 32 to close and the second valve 34 to thereafter open. Meanwhile, the second linkage 54 precludes actuation of the pump 24 until the first valve 32 is closed and the second valve 34 is opened by the first activation mechanism 16. At this point, a second fluid path is established from the pump 24 to the cannula 30 through fluid conduits 46 and 48, as well as the outlet 50. The medicament is then administered to the patient through the cannula 30.

Once the medication dosage is administered, the piston 26, and thus the second activation mechanism 18, is returned under the biasing pressure of the spring 38 to its initial position. During the travel of the piston 26 back to its first position, a given volume of the liquid medicament for the next dosage delivery is drawn from the reservoir 22 into the pump chamber 28 to provide the infusion device 10 with its next dosage delivery.

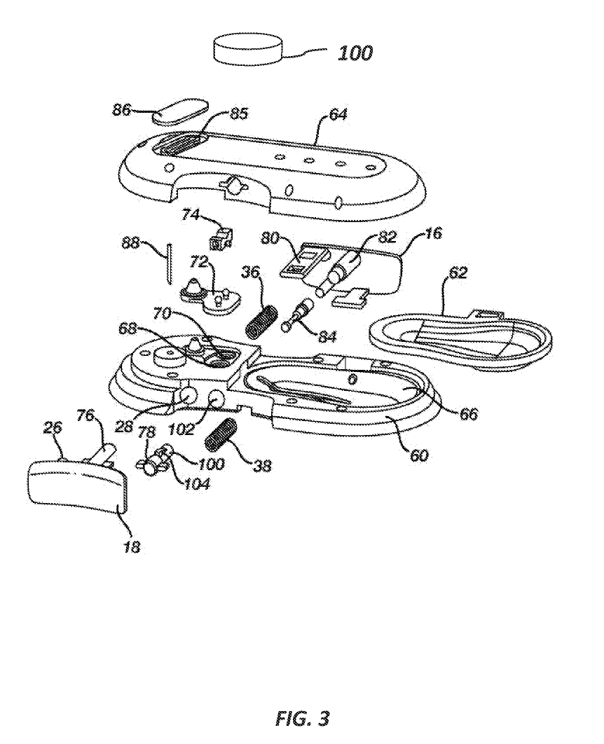

FIG. 3 is an exploded assembly view of the infusion device 10 of FIGS. 1 and 2. The main component parts include the aforementioned device layers including a base layer 60, a reservoir membrane or intermediate layer 62, and a top body layer 64. The base layer 60 is a substantially rigid unitary structure that defines a first reservoir portion 66, the pump chamber 28, and valve sockets 68 and 70 of the first and second valves 32, 34, FIG. 2, respectively. The base layer 60 may be formed of plastic, for example. The reservoir membrane layer 62 is received over the reservoir portion 66 to form the reservoir 22, FIG. 2). A valve seat structure 72 is received over the valve sockets 68 and 70 to form the first and second valves 32 and 34, FIG. 2, respectively. A rocker 74 is placed over the valve seat structure 72 in order to open and close the valves 32, 34 as will be described subsequently. The second or pump activation mechanism 18 carries the pump piston 26 that is received within the pump chamber 28. The pump activation mechanism 18 also carries a cam cylinder 76 with a lock tube 78 therein that form a portion of the second linkage 54, FIG. 2. The spring 38 returns the second activation mechanism 18 to its first position after each dosage delivery.

The first activation mechanism 16 carries a valve timing cam 80 that rocks the rocker 72. The mechanism 16 further carries a cam cylinder 82 and a cam pin 84 that is received into the cam cylinder 82. The spring 36 returns the first activation mechanism 16 to its first position after each dosage delivery. The top body layer 64 forms the top portion of the device enclosure. This layer 64 receives a planar cap 86 that completes fluid paths 85 partially formed in the top layer 64. Lastly, a needle 88 is provided that provides fluid coupling from the cannula 30, FIG. 2, to the outlet 50, FIG. 2, of the device 10.

As previously described, the infusion device 10 described herein is capable of delivering discrete doses or boluses of medication to the patient based on engagement of the first and second activation mechanisms 16 and 18. Most, if not all, patients may desire a way for their infusion device to record when a dose is delivered in a dose event. Thus, as will be further discussed below, the infusion system 10 can include a dose counter 100 in order to record dose events.

Transmitting the occurrence of each dose to a remote device such as a mobile device (e.g., a smartphone, a tablet PC, etc.) is desirable, as the structure and method for doing so minimizes the number of components that need to be added to the infusion device of FIGS. 1-3. To provide this functionality, a communication interface employing a local wireless protocol covered under relevant portions of IEEE 802.11, such as a near-field communication (NFC) interface (not shown) or other low power wireless communication links, such as Bluetooth®, Zigbee, and ANT, among others, for example, can be used to locally transmit the occurrence of each dose. Alternatively, a dose counter can provide a storage count of dose events on board the device.

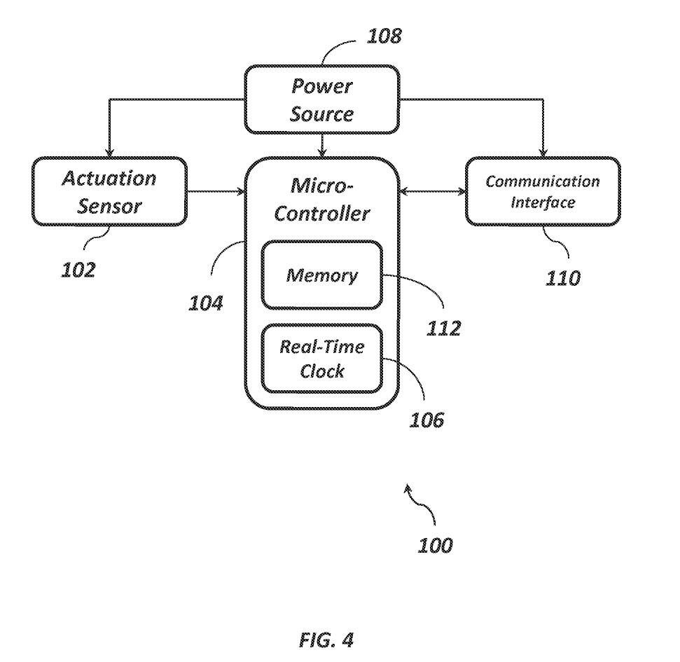

Referring now to FIG. 4, a functional block diagram of the exemplary dose counter 100 is depicted. The dose counter 100 includes an actuation sensor 102. As discussed above, the actuation sensor 102 detects a dose event by detecting movement or engagement of the activation mechanism(s) of an infusion device. In an embodiment, the activation mechanism(s), as a depressible button generally known, generates two sets of vibrations per dose event. More specifically, a first set of vibrations is produced when the button is initially depressed to create a dose event and a second set of vibrations is produced when the button is released and allowed to return to a non-activated position. Through attachment to the exterior of the housing, the actuation sensor 102 detects these vibrations as a vibration signature generated when the activation mechanism(s) of the infusion device is engaged. The actuation sensor 102 can be any suitable type of vibration sensor, such as an electret microphone, a moving coil, a moving magnet, or a piezo crystal, provided the sensor 102 is configured to detect vibrations induced by contacting the infusion device 10. As previously discussed, the sensor 102 can be attached as part of a releasable module or can be integrally supplied.

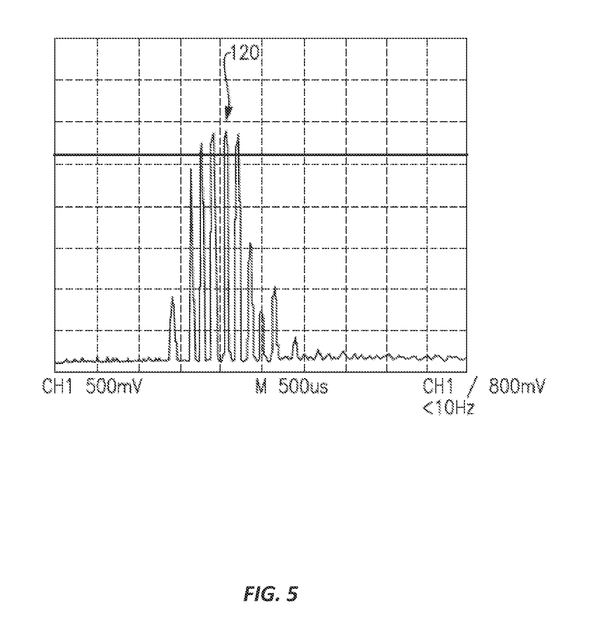

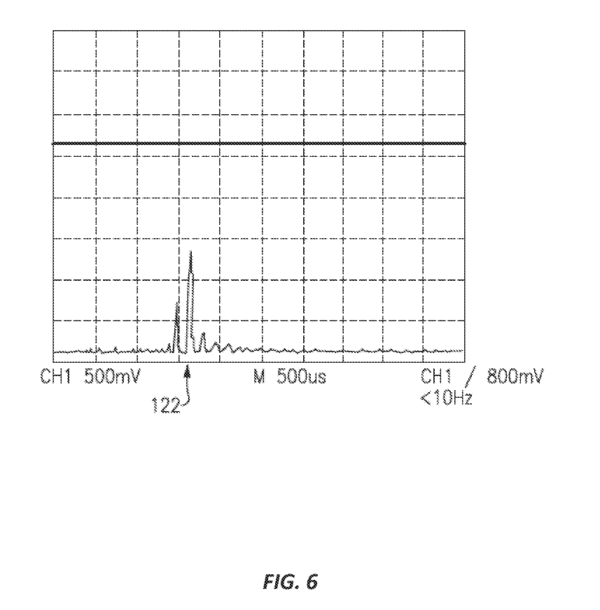

Still referring to FIG. 4, the actuation sensor 102 is coupled to a micro-controller 104. When the actuation sensor 102 detects vibrations, which may indicate engagement of the activation mechanism(s), the micro-controller 104 is configured to analyze and filter the detected vibrations. As illustrated below with regard to FIG. 5, depression of the buttons of the activation mechanism produces a signature waveform 120 having a particular shape, while incidental non-activation contact with the housing and/or buttons produces an incidental waveform 122, such as illustrated by FIG. 6, that is significantly different from the signature waveform 120 and which will be disregarded as a dose event.

The micro-controller 104 is configured to analyze the vibrations detected by the actuation sensor 102 in order to identify the waveform of the detected vibrations and deduce whether a dose event has in fact occurred. More specifically, in an embodiment, the detected waveform is compared to a stored signature waveform 120 and, if the detected waveform substantially matches the stored signature waveform 120, the micro-controller 104 identifies the detected vibrations as being representative of a dose event. In another embodiment, the micro-controller 104 compares the detected waveform to the stored parameters indicative of a vibration signature, e.g., peak amplitude, distance between peaks, etc., and, if the detected waveform substantially matches the stored parameters, the micro-controller 104 identifies the detected vibrations as being representative of a dose event. For purposes of this comparative analysis, it has been determined empirically that the time between pulses, illustrated as peaks in the detected waveform, can be measured by the micro-controller 104 and a decision made as to whether a sufficient number of pulses are detected having an amplitude and duration representative of the vibration signature.

Once the vibration signature indicative of a dose event has been identified, the micro-controller 104 is programmed to record a dose event. In one version, the micro-controller 104 can advance a counter to log the dose event. In another example, the micro-controller 104 can also store the occurrence of a dose record into resident memory 112. For purposes of storage, the memory 112 can be any suitable type of memory. For example, random-access memory (RAM) or electrically erasable programmable read-only memory (EEPROM) can be used. According to at least one version, the memory 112 can be a ferroelectric random access memory (FRAM).

According to this embodiment, the micro-controller 104 includes a real time clock 106 configured to track and maintain system time. Additionally, the clock 104 creates a timing signal used in conjunction with the vibration signature to provide a time stamp which is stored into resident memory 112.

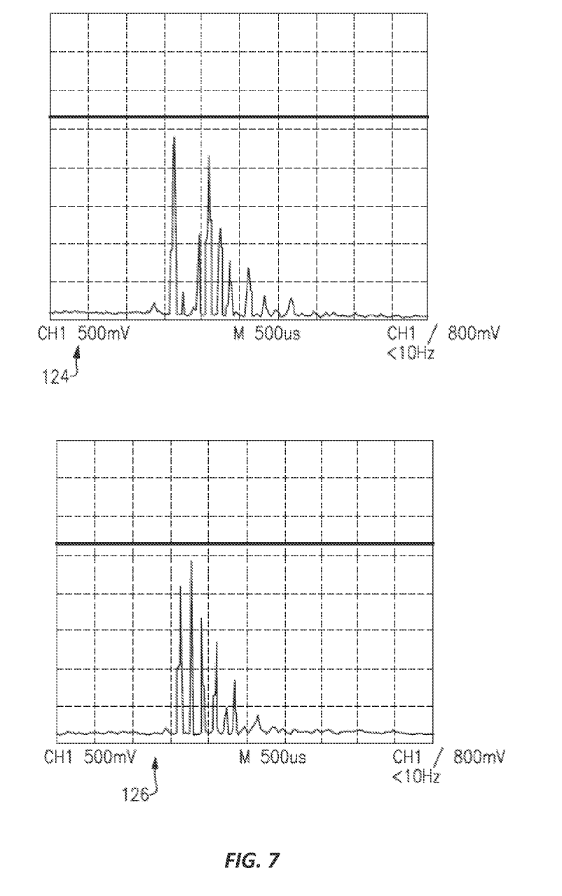

As illustrated by the waveforms 124, 126 illustrated in FIG. 7, it has been empirically determined that a signature oscillation of the vibration signature representative of a dose event of the device 10 is between 6 and 8 kHz. In order to detect this oscillation, a timer counter (not shown) of the micro-controller can be set to increment at a rate at least ten times the signature oscillation, e.g. greater than 60 kHz. Each pulse of the signature oscillation causes the instantaneous timer value to be captured and stored. After three or four captures, the micro-controller 104 compares the timer counter difference between successive peaks. If these differences fall within a predetermined range, a vibration signature is detected and a dose event is counted, along with a timestamp from the real time clock 106.

In a representative example, the clock 106 is a 100 kHz timer counter clock and the detected vibrations have a 6 kHz signature oscillation. The time of each detected peak, as well as the difference between the peaks, are indicated in the following Table 1.

TABLE 1

| Signature Peak | Time (s) | Timer Capture | Difference |
|---|---|---|---|
| 1 | 0.000167 | 16 | — |
| 2 | 0.000333 | 33 | 17 |
| 3 | 0.0005 | 50 | 17 |
| 5 | 0.000833 | 83 | 33 |
| 6 | 0.001 | 100 | 17 |
| 8 | 0.001333 | 133 | 33 |

The difference between peaks is typically 17 counts or, if a peak is missed, 33 counts. In the example illustrated by Table 1, no 4^{th} or 7^{th} peaks were detected in the signature oscillation. Allowable values of the difference values for signature oscillations ranging from 6 kHz to 8 kHz are from 17 to 12 counts, respectively. In this example, a dose event is recorded if three difference measurements exist, each falling between the allowable count range (i.e., 12 to 17) or double this count range (i.e., 24 to 34), the latter accounting for any missed peaks.

Returning to FIG. 4, a communication interface 110 is coupled to the micro-controller 104. Using this communication interface 110, the micro-controller 104 can transfer recorded dose event information to another device, such as a smartphone (not shown). In an example, the micro-controller 104 transfers a dose event record each time a record is generated. In another example, the micro-controller 104 can transfer dose event records on demand when another device initiates communication with the micro-controller 104 via the communication interface 110. The communication interface 110 can employ a conventional wireless protocol with a remote mobile device (not shown). For example, the communication interface 110 can employ a near field communication (NFC) or other low power wireless protocol, including Bluetooth, Zigbee, and ANT among others. Alternatively, a hard-wired connection could be provided between the infusion device and the other device.

According to this embodiment, a power source 108 powers each of the micro-controller 104 and actuation sensor 102. The power source 108 can be any suitable type of power source, such as a lithium or alkaline battery. In another example, the power source 108 can be an energy generator that harvests energy produced by the mechanical operation of the infusion device. While the power source 108 is illustrated herein as part of the dose counter 100, the power source 108 could alternatively be incorporated directly in the infusion device, rather than the dose counter 100.

As noted above, when the activation mechanism is a depressible button, actuating the depressible button generates two sets of vibrations per dose. In order to prevent the dose counter from counting both sets of vibrations as a dose event, thereby double counting a single dose, the micro-controller 104 can initiate a temporary counting lock-out circuit (not shown). The lock-out circuit prevents the micro-controller 104 from logging a dose event when the lock-out circuit is active. The lock-out circuit is operable for a short period of time, such as 500 ms, after a dose event is identified, thereby preventing a single dose event from being counted more than one time.

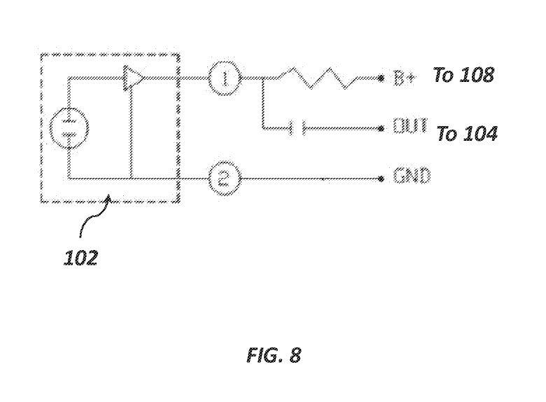

FIG. 8 illustrates a schematic diagram of an exemplary actuation sensor 102. The sensor 102, which can be an electret microphone according to this example, can include a resistor 130, such as a 100 Kohm resistor. Additionally, the sensor 102 can optionally include a capacitor 132 disposed in series with the output. For example, the capacitor 132 can be included if the circuit is used as an audio microphone. Because the sensor 102 consumes low power, such as less than 30 μA, the sensor 102 can be powered by a small power source, such as a battery. The actuation sensor 102 can be coupled to a power source 108, such as a 3V CR2032 battery, and to the micro-controller 104. Optionally, the actuation sensor 102 can include a second metal oxide semiconductor field-effect transistor (MOSFET) (not shown) to increases the sensitivity of the actuation sensor 102. This second MOSFET can be used in order to convert the vibration signature into a purely binary signal.

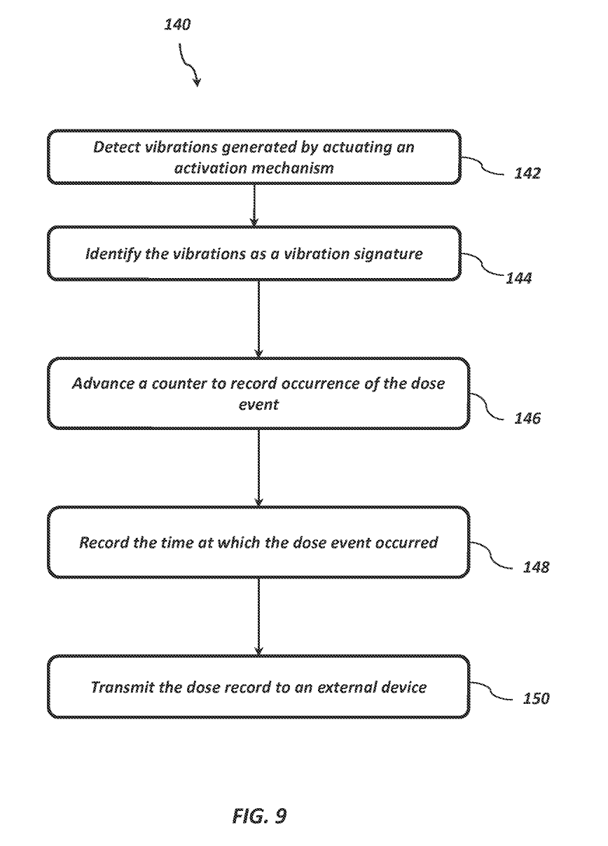

Referring to FIG. 9, an exemplary method 140 for counting a dose event of an infusion device is described. As described above, the infusion device is mechanically operated and includes a pump and at least one mechanical activation mechanism for engaging the pump in order to cause a dose event to administer a medicament, such as insulin, to a patient. Though not shown, in one version the dose counter can initially be attached to the housing of the infusion device in the event a dose counter is not already present. At block 142, vibrations generated by actuating the at least one activation mechanism are detected by an actuation sensor. At block 144, a micro-controller coupled to the actuation sensor analyzes the detected vibrations and determines whether the vibrations constitute a vibration signature, indicative of a dose event. To accomplish this determination, the micro-controller analyzes the detected vibrations as described above with regard to FIG. 4 and determines whether the vibrations detected are representative of a dose event. At block 146, the micro-controller advances a counter to record occurrence of the dose event indicated by the vibration signature. At block 148, the micro-controller also records a timestamp of the dose event. At block 150 and according to one version, the micro-controller transmits the dose record, including the timestamp, to an external device via a communication interface. Alternatively, the dose counter module or device could include an integral display (not shown). The external device can store the transmitted dose record and/or display the transmitted dose record on a display device.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” ‘subsystem” and/or “system.” Furthermore, aspects of the present invention may take the form of a computer program product embodied in one or more computer readable medium(s) having computer readable program code embodied thereon.

### PARTS LIST FOR FIGS. 1-10

10 infusion device
12 enclosure
14 base
16 first activation mechanism
18 second activation mechanism
20 fill port
22 reservoir
24 pump
26 pump piston
28 pump chamber
30 cannula
32 first valve
34 second valve
36 spring
38 spring
40 fluid conduit
42 fluid conduit
44 fluid conduit
46 fluid conduit
48 fluid conduit
50 outlet
52 first linkage
54 second linkage
60 base layer
62 reservoir membrane layer
64 top body layer
66 reservoir portion
68 valve socket
70 valve socket
72 valve seat structure
74 rocker
76 cam cylinder
78 lock tube
80 valve timing cam
82 cam cylinder
84 cam pin
85 fluid paths
86 planar cap
88 needle
100 dose counter
102 actuation sensor
104 micro-controller
106 real-time clock
108 power source
110 communication interface
120 signature waveform
122 incidental waveform
124 waveform
126 waveform
130 resistor
132 capacitor
140 method
142-150 method blocks

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An infusion device, comprising:
a reservoir that holds a liquid medicament;
a pump that displaces a volume of the liquid medicament when mechanically activated by an activation mechanism to deliver a dose of the liquid medicament and thereby signifying a dose event; and
a dose counter, comprising:
a sensor configured to detect vibrations indicative of operation of the activation mechanism.

2. The infusion device of claim 1, the dose counter further comprising a micro-controller coupled to the sensor to record the dose event.

3. The infusion device of claim 1, wherein the activation mechanism comprises at least one depressible button.

4. The infusion device of claim 3, wherein the at least one depressible button generates a vibration signature indicative of a dose event.

5. The infusion device of claim 4, wherein the micro-controller is configured to distinguish the vibration signature from an incidental vibration.

6. The infusion device of claim 2, wherein the micro-controller comprises a real-time clock.

7. The infusion device of claim 2, wherein the micro-controller further comprises a memory to store a record and a timestamp of each dose event.

8. The infusion device of claim 1, wherein the micro-controller is configured to prevent operation for a predetermined period of time after determination of the dose event in order to prevent counting the dose event more than once.

9. The infusion device of claim 1, further comprising a portable power device.

10. The infusion device of claim 1, the dose counter further comprising a communication interface configured to transmit the time of each activation of the pump.

11. The infusion device of claim 18, wherein the dose counter is removably coupled to the infusion device.

12. A dose counter for a mechanically operable infusion device, said infusion device comprising a pump and at least one mechanical activation mechanism for engaging the pump to deliver a dose and signify a dose event, the dose counter comprising:
at least one sensor configured to detect vibration indicative of the dose event; and
a micro-controller comprising:
a clock; and
a memory,
the micro-controller configured to record occurrence of each dose event and a time of each dose event in the memory.

13. The dose counter of claim 12, further comprising a portable power source configured to power the dose counter.

14. The dose counter of claim 12, wherein the at least one sensor comprises an electret microphone.

15. The dose counter of claim 12, wherein the dose event generates a vibration signature that can be detected by the at least one sensor.

16. The dose counter of claim 12, further comprising a communication interface, the micro-controller being configured to transmit a record of each dose event using the interface.

17. The dose counter of claim 16, wherein the communication interface utilizes a low power wireless protocol.

18. The dose counter of claim 12, the micro-controller further comprising a counter configured to record occurrence of each dose event.

19. A method for using an infusion device, said infusion device comprising a pump, at least one mechanically actuated mechanism for engaging the pump to cause a dose event, and a sensor, the method comprising:
detecting vibrations using the sensor generated by engaging the at least one activation mechanism to cause the dose event; and
determining whether the vibrations constitute an actual dose event.

20. The method of claim 19, further comprising advancing a counter to record occurrence of the dose event.

21. The method of claim 19, further comprising recording a timestamp of the dose event.

22. The method of claim 19, wherein the sensor is coupled to a micro-controller configured to record a dose event, the micro-controller being configured to distinguish detected vibrations for determining the occurrence of a dose event.

23. The method of claim 22, wherein the sensor comprises an electret microphone.

24. The method of claim 19, further comprising transmitting the recorded time of the dose event.

25. The method of claim 22, wherein the micro-controller is configured to engage a lock-out protocol to prevent counting a dose event more than once.

26. The method of claim 19, wherein the activation mechanism comprises at least one depressible button.

27. The method of claim 26, wherein the at least one depressible button generates a vibration signature indicative of a dose event.

28. The method of claim 19, wherein the sensor is removably coupled to the infusion device.

29. The method of claim 19, wherein the sensor is integral to the infusion device.
